(19) United States
(12) Patent Application Publication
(10) Pub. No.: US 2023/0317284 A1
(43) Pub. Date: Oct. 5, 2023

(54) USING FOURIER APPROXIMATIONS TO CREATE DECISION BOUNDARIES IN MACHINE LEARNING

(71) Applicant: KONINKLIJKE PHILIPS N.V., EINDHOVEN (NL)

(72) Inventors: Serverius Petrus Paulus PRONK, VUGHT (NL); Johannes Henricus Maria KORST, EINDHOVEN (NL)

(21) Appl. No.: 18/020,095

(22) PCT Filed: Aug. 5, 2021
(86) PCT No.: PCT/EP2021/071848

§ 371 Date: Feb. 7, 2023

Related U.S. Application Data
(60) Provisional application No. 63/064,955, filed on Aug. 13, 2020.

(51) Int. Cl. G16H 50/20; G16H 50/70

(52) U.S. Cl. CPC G16H 50/20; G16H 50/70

(57) ABSTRACT

A method (100) of generating a machine-learned (ML) decision tree classifier (12) includes: iteratively adding child nodes to nodes of the ML decision tree classifier by: selecting a plurality of features from the set of features; creating one or more boundaries (34) in a plane (30) defined by the selected plurality of features, the one or more boundaries partitioning the plane into regions (36) that split the training data associated to the node into at least two subsets of training data, the one or more boundaries being created based on the class labels of the training data associated to the node; for each subset of training data, adding to the node a child node having associated training data consisting of the subset of training data; and defining a classification rule for the node using the created one or more boundaries in the plane.

### FIELD

The following relates generally to the machine learning classifier arts, fault finding arts, medical computer aided diagnosis (CADx) system arts, medical device maintenance arts, decision tree classifier visualization arts, decision tree classifier navigational arts, and related arts.

### BACKGROUND

Classifiers are used in many tasks, such as CADx systems, troubleshooting systems, recommender systems, and so forth. As one example, a decision tree may be designed as a fault-finding tree for guiding a service technician in diagnosing a failure of a medical imaging device or other complex equipment. In such an application, the input to the decision tree is values of a set of features for the medical device under diagnosis. For example, the features may include results of diagnostic tests, symptoms of the failure, and/or so forth. The output of the decision tree is then a proposed root cause of the failure and/or a proposed solution for the failure. As another example, a CADx system is designed to classify whether a patient may have a particular medical condition (binary classifier), or which of a set of possible medical conditions is most likely (multi-label classifier). In a CADx system, the patient is represented by a set of features, such as vital sign readings (e.g., heart rate, blood pressure, respiratory rate, SpO₂ reading), demographic features (e.g., gender, age, ethnicity), test results (e.g., hematology test results, features determined by medical imaging), and/or so forth. The CADx classifier is trained on training data of historical patients represented by sets of features, with the historical patients labeled by their known medical diagnoses (typically provided by the patient's physician).

Usually, the root cause or solution generated by a fault-finding tree applied for diagnosing a fault in a complex machine is only a proposal, and the service technician ultimately decides how to proceed with the repair. Likewise, the output of the CADx system is only a proposed diagnosis that is considered by the patient's physician, along with other information, along with the physician's expertise and possibly in consultation with physician colleagues, in order to diagnose the patient. Fault finding trees for diagnosing faults in complex equipment are challenging to construct, because various symptoms, diagnostic test results, and/or so forth may be variously correlated or anti-correlated with various possible root causes/solutions. Likewise, CADx classifiers are challenging to construct, because most patient features are positively or negatively correlated with multiple candidate conditions, and it is only by consideration of combinations of patient features that a CADx classifier can provide a reasonably reliable proposed diagnosis.

In performing machine learning to train a classifier, the basic input for learning (i.e., training) the classifier is a dataset that contains a large number of labeled instances (e.g., persons, objects, and so forth; specifically data from past imaging device service calls in the illustrative case of fault finding tree training; or historical patients in the illustrative example of training a CADx classifier). Each instance is described by a multitude of feature values and the labels belong to a typically small, discrete set. A feature may describe any aspect of an instance, such as color, shape, size, age, weight, etc., and it is often of a numeric character, although features may be of other data types such as Boolean values, text strings, or so forth. The label set can be as simple as {0, 1}, standing for any dichotomy, like a negative of positive outcome of a medical test, a dislike or like of a particular movie, and so forth. It can also contain more than two elements, such as, for example, {dog, cat, horse, cow, bird}, indicating the type of animal that an input picture shows. Once the algorithm has made a model of the input dataset, it aims to classify similar, but usually different instances than those in the input dataset, by assigning a label to each of them.

Decision trees are popular machine learning algorithms for binary classification, partly due to their relative simplicity. A decision tree is built by repetitively subdividing the dataset into subsets using a threshold on the values of one of the features, ideally in such a way that the final subsets contain only instances of one class. At the split of a subset S, one feature is chosen and a threshold for this feature, whereby all instances in S with a value for this feature at most this threshold is separated from the others in 5, thus creating two subsets. The choice of which feature and threshold to use for the split is governed by an optimality criterion, such as, for example, the Gini diversity index. When a subset contains instances of only one class, this subset is not further split anymore. Additional stopping criteria are typically used to stop the splitting process, for example, until a next split would result in too small subsets or until a maximum number of levels has been reached.

If the features are represented as vectors of length M, then this way of creating a decision trees successively splits the entire feature space along hyperplanes of dimension M−1 that are parallel to M−1 axes. For example, in the simple example in which the set of features includes only two features f₁ and f₂, then a split of the two-dimensional plane into smaller sub-planes uses lines that are parallel to one of the axes. For an example in which the set of features includes only three features, a split of the three-dimensional space into smaller sub-spaces uses planes corresponding to values of a single feature. For higher-dimensional spaces, the split is into hyperplanes corresponding to values of a single feature.

The following discloses certain improvements to overcome these problems and others.

### SUMMARY

In one aspect, a non-transitory computer readable medium stores instructions executable by at least one electronic processor to perform a method of generating a machine-learned (ML) decision tree classifier. The method includes: iteratively adding child nodes to nodes of the ML decision tree classifier starting at a root node having associated training data represented by values of a set of features and labeled with class labels. The addition of child nodes to a node includes: selecting a plurality of features from the set of features; creating one or more boundaries in a plane defined by the selected plurality of features, the one or more boundaries partitioning the plane into regions that split the training data associated to the node into at least two subsets of training data, the one or more boundaries being created based on the class labels of the training data associated to the node; for each subset of training data, adding to the node a child node having associated training data consisting of the subset of training data; and defining a classification rule for the node using the created one or more boundaries in the plane. The method further includes at least one of (i) storing the ML decision tree classifier in a database and (ii) for at least one iteration of the iterative adding, displaying the plane with the created one or more boundaries on a display device.

In another aspect, an apparatus for generating a ML decision classifier includes at least one electronic processor programmed to: select two features from a set of training data; generate a plane using the selected plurality of features, wherein the data in the set of training data is represented in the plane as points; create polygons in the plane to generate the ML decision classifier, the one or more boundaries delineating a class of the training data; and at least one of store the ML decision classifier in a database and display the plane with the created boundaries on a display device.

In another aspect, a method of generating a ML decision tree classifier includes: selecting two features from a set of training data; generating a plane using the selected plurality of features, wherein the data in the set of training data is represented in the plane as points; creating polygons in the plane to generate the ML decision classifier, the one or more boundaries delineating a class of the training data; performing a smoothing operation on boundaries of the created polygons using a Fourier analysis; and at least one of storing the ML decision classifier in a database and displaying the plane with the created boundaries on a display device.

One advantage resides in providing a decision tree classifier with planes that do not need to be parallel to the axes thereof.

Another advantage resides in providing a decision tree classifier with shapes that do not require straight edges.

Another advantage resides in splitting of a two-dimensional plane of a decision tree classifier being defined by pairs of features, rather than a single feature.

Another advantage resides in using machine-learning processes to split a two-dimensional plane of a decision tree classifier.

Another advantage resides in improved accuracy of a decision tree classifier, with overfitting being reduced.

Another advantage resides in allowing a user of the decision tree classifier to understand how the given set of instances is approximated and to inspect where errors are made in order to further improve performance.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an apparatus for navigating a hierarchical electronic questionnaire in accordance with the present disclosure.

FIGS. 2-5B show examples of decision tree classifier generated by the apparatus of FIG. 1.

### DETAILED DESCRIPTION

The following relates to an improvement in training of decision tree-type classifiers. In a conventional decision tree, each branch point of the tree splits the training data into two parts on the basis of thresholding a feature value. The branch point breaks the training data flowing into the branch point into two groups, and the threshold is chosen so as to maximally segregate two labeled classes. By repeating this process for several branch points, the goal is that at each leaf node will contain only training instances of a single labeled class.

Conventional tree-type classifiers have disadvantages. The hyperplanes defined at each branch point are parallel to M−1 axes of the M-dimensional hyperspace, and the hypercubes in the M-dimensional feature space have straight edges. The subdivision of the feature space in a conventional decision-tree classifier employs hyperplanes that are parallel to M−1 axes. Furthermore, the hypercubes formed in this way contain straight edges. These properties may not be realistic, i.e., need not be present in the dataset. For example, if there are two classes and they are separated by a line f₂−f₁=c, then making a proper decision tree with only horizontal or vertical boundaries is quite cumbersome.

To improve on this situation, the following discloses that at each branch point the division of the data into two parts is done on the basis of two features, rather than only a single feature. For a designated chosen two features f₁ and f₂ (where the subscripts “1” and “2” merely designate chosen first and second features respectively, and do not for example require that these chosen features be the first and second elements of an ordered feature vector), the threshold of the conventional decision tree is replaced by one or more closed plane figures defined in the f₁−f₂ plane. The plane figures are first defined as polygons using points delineated on lines connecting data points of opposite label, and then a Fourier analysis is performed to smooth the boundaries. The disclosed systems and methods use a using Voronoi diagram analysis to define the polygons. More generally, a plane partitioning algorithm is run (analogous to image segmentation) to delineate regions that are (at least mostly) of one class in the f₁−f₂ plane. More generally, a smoothing algorithm may be applied to smooth the boundaries of the defined regions. In an alternative embodiment, spline curve fitting is employed to define the regions; however, this is noted to be computationally more complex.

In some embodiments disclosed herein, the process is applied for training a multi-class classifier. This is done by training each branch point to perform a one-against-all classification for one class, and iterating between different classes for subsequent branch points.

In other embodiments disclosed herein, a Principal Component Analysis (PCA) is performed to define the features f₁ and f₂ as PCA features. This could be done in defining the feature vectors prior to training the classifier, or during the classifier training process.

The disclosed systems and methods can be used for practical applications, for example to be used in diagnostic support systems utilized by service technicians diagnosing faults with complex equipment, or Computer-Aided Diagnosis (CADx) systems, or the like.

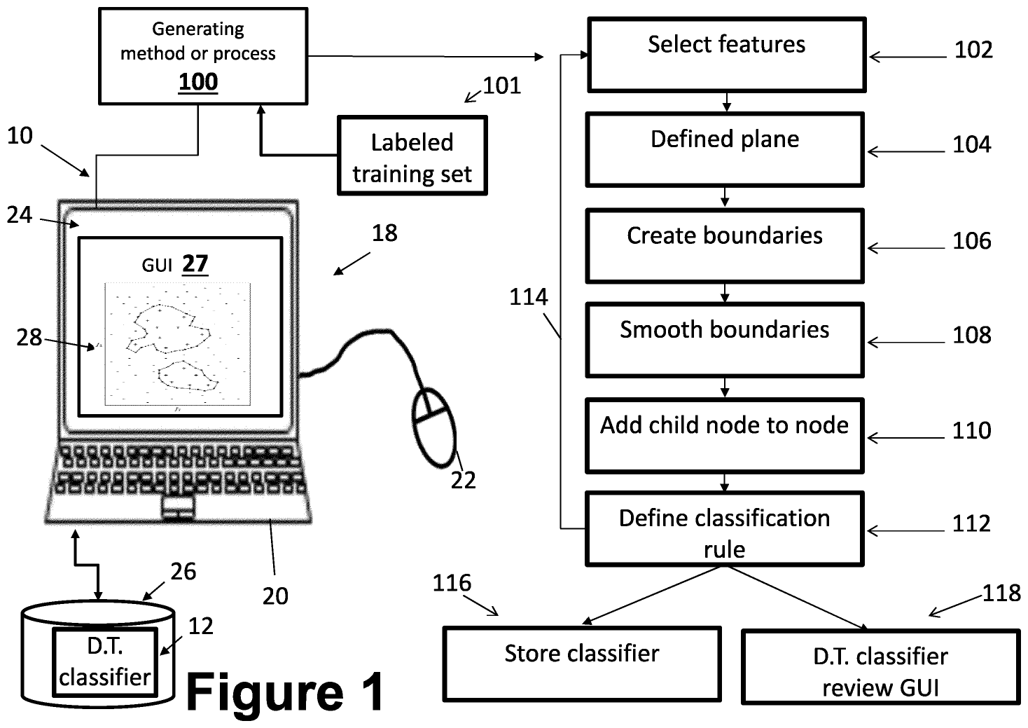

With reference to FIG. 1, an illustrative apparatus 10 for generating a machine-learned (ML) decision tree classifier 12 is shown. FIG. 1 also shows an electronic processing device 18, such as a workstation computer, tablet computer, cellular telephone (“cellphone”), or more generally a computer. The disclosed decision tree classifier generating process may be performed entirely by a local electronic processor, or a portion of the decision tree classifier generating process may be performed by a remote electronic processor. In the latter case, the electronic processing device 18 is at least partially embodied as a server computer or a plurality of server computers, e.g. interconnected to form a server cluster, cloud computing resource, or so forth. The workstation 18 includes typical components, such as an electronic processor 20 (e.g., a microprocessor; again, in some embodiments part of the decision tree classifier generating process may be performed by the microprocessor of a remote server or cloud computing resource), at least one user input device (e.g., a mouse, a keyboard, a trackball, touch-sensitive display, and/or the like) 22, and at least one display device 24 (e.g. an LCD display, plasma display, cathode ray tube display, and/or so forth, which optionally may be a touch-sensitive display thus also serving as a user input device). In some embodiments, the display device 24 can be a separate component from the workstation 18.

The electronic processor 20 is operatively connected with one or more non-transitory storage media 26 which stores the decision tree classifier 12. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 27 for display on the display device 24. The decision tree classifier 12, once generated, is displayed on the display device 24 via the GUI 27. In addition, the non-transitory computer readable medium 26 stores information related to the generation of the decision tree classifier 12.

With continuing reference to FIG. 1, the at least one electronic processor 20 is configured as described above to perform a method or process 100 for generating the decision tree classifier 12. The non-transitory storage medium 26 stores instructions which are readable and executable by the at least one electronic processor 20 to perform disclosed operations including performing the method or process 100. In some examples, the method 100 may be performed at least in part by cloud processing.

In FIG. 1, an illustrative embodiment of method 100 is diagrammatically shown as a flowchart. The method 100 receives a labeled training set 101 of instances (which can be stored in the non-transitory computer readable medium 26), with each instance represented by values for a set of features and labeled with a label selected from a set of labels. In the illustrative example of training a fault finding tree for diagnosing a fault in a medical imaging device, the instances of the labeled training set 101 are suitably data from diagnostic sessions of past historical service calls represented by values for a set of features, where the features may for example include symptoms of the failure and/or diagnostic test results run by the imaging device operator or the service technician. In the illustrative example of training a CADx classifier for classifying whether a patient has a particular medical condition (e.g., a particular disease), the instances of the labeled training set 101 are historical patients represented by values for a set of features. The historical patients of the training set 101 include patients who were diagnosed by a physician with the medical condition (positive examples) and patients who were examined by a physician but were not diagnosed with the medical condition (negative examples). The set of features may include features such as vital sign readings (e.g., heart rate, blood pressure, respiratory rate, SpO₂ reading), demographic features (e.g., gender, age, ethnicity), test results (e.g., hematology test results, features determined by medical imaging), and/or so forth. The set of labels includes two values, e.g. represented as the set {0, 1} where the label 0 denotes the patient was not diagnosed with the medical condition while the label 1 denotes the patient was diagnosed with the medical condition. It is to be appreciated that this is an illustrative example; more generally, the disclosed method 100 can be used to train a classifier for any task.

The method 100 generally includes iteratively adding child nodes to nodes of the ML decision tree classifier 12, starting at a root node having associated training data represented by values of a set of features and labeled with class labels. One iteration of the method 100 includes the diagrammatically depicted operations 102, 104, 106, 108, 110, 112, resulting in the generating of a new node of the decision tree classifier with an associated classification rule. Process flow then returns via loop-back arrow 114 to perform the next iteration which adds another node to the decision tree classifier. The iterative method 100 thereby “grows” branches of the decision tree classifier. Each added node defines a split, and such splitting continues for each branch path until a leaf node is reached at which the two portions produced by the split each contain instances with a single class label (for example, at the leaf one portion contains all examples labeled with 0 and the other portion contains all examples labeled with 1; or at least, the one portion contains mostly instances labeled 0 and the other contains mostly instances labeled 1, with the mislabeled instances in each portion being below some acceptable classification error). For a given iteration, at an operation 102, a plurality of (that is, at least two) features is selected from the set of features. The choice of which two features to use for the split may be based on any suitable optimality criterion such as those commonly used in conventional decision tree learning, but extended to select two features rather than just a single feature as in conventional decision tree learning. By way of non-limiting illustrative example, the selection of the features (which are designated herein without loss of generality as features f₁ and f₂) can be based on the Gini diversity index.

It is to be appreciated that a Principal Component Analysis (PCA) process can optionally be performed. In one approach, this is performed on the set of features prior to initiation of the method 100, so that the set of features comprises PCA features. In another approach, the PCA is performed on a per-iteration basis as part of operation 102 to define the chosen features f₁ and f₂. In this case, the features f₁ and f₂ are suitably chosen as the first and second principal components produced by the PCA analysis. Additionally, the classification rule (defined in operation 112 to be described) can include computing PCA features f₁ and f₂.

At an (implied) operation 104, a plane is defined by the selected features, that is, f₁ and f₂ plane. The data in the training data associated to the node is represented in the plane as points.

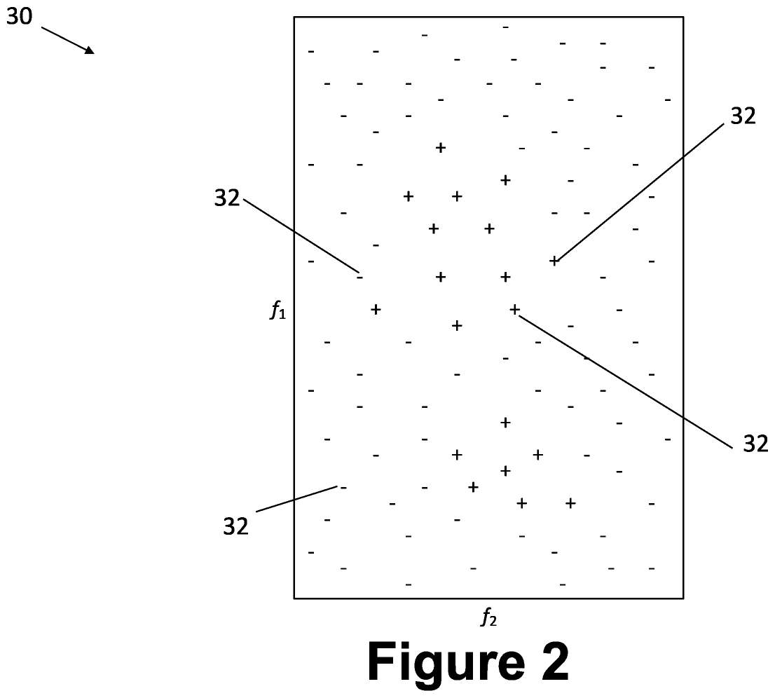

FIG. 2 shows an example of the operations 102 and 104. For each subset S of the training data (starting with the complete dataset) the splitting process starts with choosing two features f₁ and f₂ in operation 102. This may be done in various ways. In one particular example, in line with how a single feature is chosen in conventional decision trees, all possible combinations (f₁, f₂) of two different features are considered. A feature combination is chosen that is optimal in the operation 102, using an optimality criterion. As shown in FIG. 2, this results in the (implied) operation 104 in which a plane 30 is defined by the features f₁ and f₂. A plurality of points 32 are represented in the planes. The points 32 represent instances of the training set 101 with the point for each instance being at its (f₁, f₂) coordinate in the plane 30. The points 32 are represented in FIG. 2 using the class labels of the instances of the training data 101, and these are represented in illustrative FIG. 2 as positive (“+”) or negative (“−”) labels. (For example, in the illustrative example of training a fault finding tree, the “+” symbol may represent the label 1 indicating the past historical imaging device diagnostic session was diagnosed with a given root cause; while the “−” symbol may represent the label 0 indicating the past historical imaging device diagnostic session was not diagnosed with a given root cause. In the illustrative example of training a CADx classifier, the “+” symbol may represent the label 1 indicating an instance (historical patient) labeled as having been diagnosed with the medical condition; while the “−” symbol may represent the label 0 indicating an instance (historical patient) labeled as diagnosed as not having the medical condition.

At an operation 106, one or more boundaries 34 are created in the plane 30. The boundaries 34 partition the plane 30 into regions 36 that split the training data associated to the node into at least two subsets of training data. The boundaries 34 are created based on the class labels of the training data associated to the node of the decision tree classifier 12.

In one example, the boundaries 34 are created as splines in the plane 30. In another, more particular example, the boundaries 34 are created as polygons 38 in the plane 30. The points 32 having different class labels (e.g., positive or negative) can be connected with lines 40. This can be performed using, for example, a Voronoi diagram analysis on the points 32 in the plane 30 to generate the polygons 38.

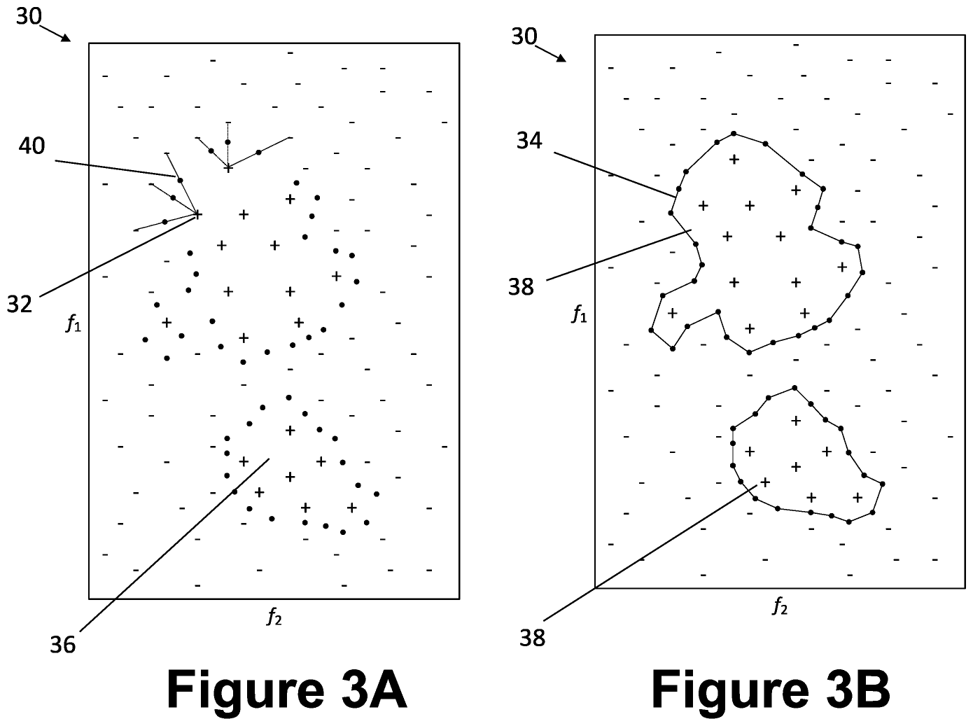

FIGS. 3A and 3B show an example of the operation 106. Generally, all the instances in each subset S of the training data have different classes (e.g. positive or negative, or more than two classes). To create an initial set of boundaries 34 around, for example, the positive classes, the Voronoi diagram is used (which is a known algorithm to isolate points in a plane) to identify neighboring positive and negative class label points 32. For each pair of positive-negative neighboring points 32, a line 40 can be drawn, as shown in FIG. 3A.

The points 32 can be interconnected with lines 40 to form the polygons 38, as shown in FIG. 3B. These polygons 38 constitute the initial set of boundaries. In some examples, if a point 32 having a positive case label has no neighbors with a negative case label, but is close to a surrounding polygon 38, additional lines 40 and points 32 can be drawn between the polygon and the positive label point 32. In other examples, when many small polygons 38 are created, the smallest ones may be ignored, causing the points 32 with positive labels to become part of a region with points with negative labels, or vice versa. Although this can increase the Gini diversity index in the resulting subdivision, the problem of overfitting may be alleviated. In some examples, a user can use the at least one user input device 22 of the workstation 18 to select positions of the lines 40 within the plane 30.

At an operation 108, a smoothing operation is performed on the created boundaries 34. For example, the smoother operation can include a Fourier analysis on the boundaries 34. The generated polygons 38 can have irregular shapes with sudden turns, which can result in overfitting the data. The smoothing operation can include a Fourier analysis.

To do so, the (f₁, f₂) plane 30 is viewed as the complex plane. The polygon(s) 38 are viewed as a complex function on, for example, e.g., a real number interval [0,1], where ƒ(0)=ƒ(1), (i.e., a closed function). Based on Fourier analysis, this function ƒ can be described by the following, infinite series according to Equation 1:

ƒ(x)=Σ_{n=-∞}^{+∞}cₙ·e^{2πinx}, (1)

and the complex Fourier coefficients cₙ are defined according to Equation 2:

cₙ=∫₀¹ƒ(x)·e^{-2πinx}dx (2)

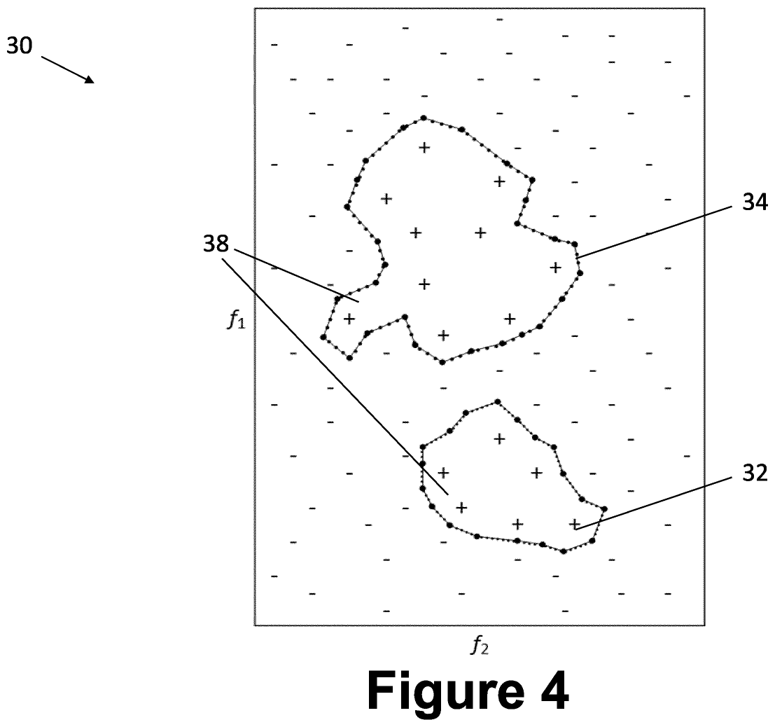

for n=−∞ . . . , ∞. For the calculation of the coefficients, a numerical integration can be performed, for which the polygon 38 needs to be described in more detail than only the given points. One possibility is to sample the polygon at equidistant spaces along the polygon, as shown in FIG. 4, where, for illustrative purposes, only 100 samples are chosen per polygon. In practice, this number may be chosen much higher in order to obtain good approximations of the coefficients and may differ per polygon 38.

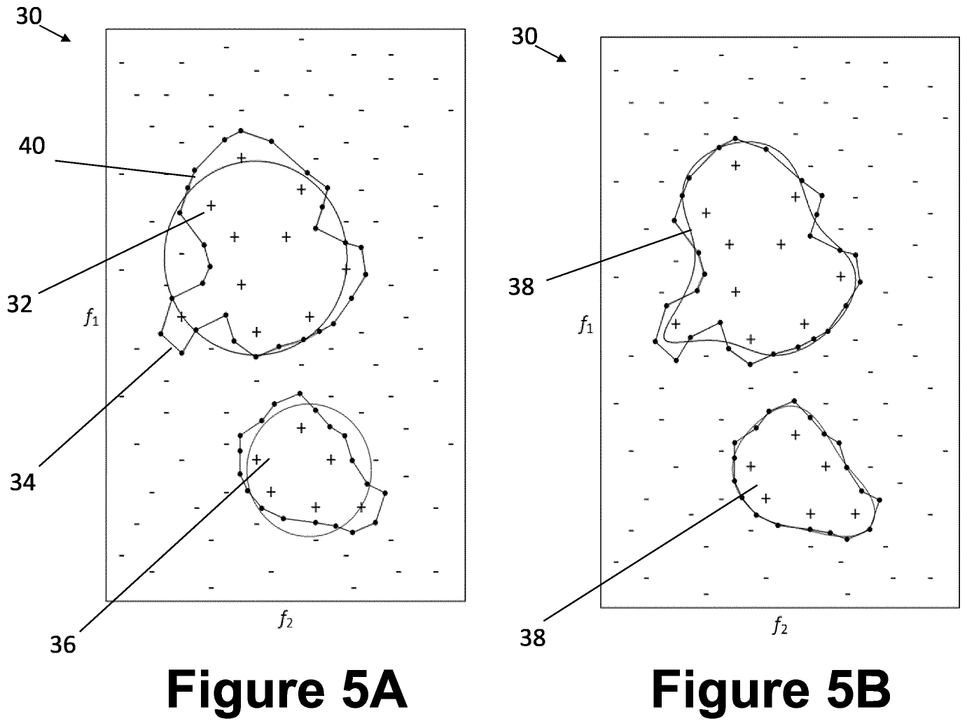

The function ƒ can be approximated by using only a finite number of terms of the infinite series. For example, by only using n=0, 1, a circular approximation of the polygons 38 is obtained, as shown in FIG. 5A. By incorporating more terms, the approximation becomes more accurate, as shown in FIG. 5B, where the range n=−4, . . . , +4 is used. Per polygon 38, a different range can be chosen, depending on the sum of squared errors between the polygon and the smoothing function.

As the approximation is a smooth, differential function, it can optionally be approximated again by a suitable polygon 38 in order to speed up the decision of whether a chosen point 32 is inside or outside this suitable polygon as a proxy for the decision of whether this point is inside or outside the smooth approximation. Also, the accuracy with which this suitable polygon 38 approximates the smooth approximation can be tuned.

This method of splitting and approximating can be repeated for the created subsets, typically using a different pair of features to further split each of the created subsets in smaller sets, by which the Gini diversity index is further decreased.

Instead of polygons 38, splines could be used, although these typically require many more parameters to be estimated, leading to a significantly more complex model than when a Fourier series is used, where only the number of Fourier coefficients is used as a parameter. The machine learning algorithm (e.g., a support vector machine) can be extended using the kernel trick, may generate irregular decision boundaries 34, but these algorithms are typically much less intuitive and insightful and do not provide means to control the false positives and false negatives at this level of detail.

The case of more than two classes can be considered in much the same way, by iteratively isolating one class c from the others, whereby elements of class c that are not isolated are ignored in the following iterations. Eventually, these elements can add to the Gini diversity index. This repetition can be done while subdividing one node into at most as many nodes as there are classes, or by successively creating additional levels in the tree using binary splitting as in the two-class case. In the former case, the boundaries 34 that are created fulfill a similar role as the surrounding rectangle in subsequent iterations.

By adapting the decision boundary as described above, the generalizability, and thus the accuracy of the machine learning problem can be improved, and overfitting reduced. Furthermore, the transparency of the method is a clear advantage, as the user will be able to understand how the given set of instances is approximated and to inspect where errors are made in order to further improve performance.

At an operation 110, for each subset of training data, a child node having associated training data consisting of the subset of training data is added to the node of the decision tree classifier 12. Since the data are split into two subsets, this results in adding two child nodes. For example, referring back to FIG. 4, the child node representing the “+” class will include the subset of data inside the polygons 38; while the child node representing the “−” class will include the subset of data outside of the polygons 38. At an operation 112, a classification rule for the node is defined using the boundaries 34 in the plane. Process flow then returns via flow arrow 114 to perform the next iteration, thereby iteratively adding child nodes to nodes of the ML decision tree classifier (starting at a root node) in order to iteratively build the decision tree classifier 12.

In some embodiments, at an operation 116, the generated decision tree classifier 12 is stored in a database (e.g., the non-transitory computer readable medium 26). In this embodiment, the generated decision tree classifier 12 can be retrieved from the database 26, and used to classify an input (e.g., from the user) represented by values of the set of features using the classification rules of the retrieved ML decision tree classifier.

In other embodiments, at an operation 118, for at least one iteration of the iterative adding, a decision tree review GUI 28 is provided which displays the plane 30 with the boundaries 34 on the display device 24. In this embodiment, the GUI 27 is provided on the display device, and optionally the review GUI 28 allows for the boundaries 34 to be manually adjusted to generate user-adjusted created boundaries. For example, the user can click on a portion of a boundary 34 using a mouse and drag the boundary to adjust its position. For the at least one iteration, the defining of the classification rule uses the user-adjusted created one or more boundaries.

The decision tree classifier 12 can be used for any suitable purpose, such as in the case of a CADx classifier for diagnosing a patient. In such an example, the training data comprises patients represented by the set of features including patient features, the class labels comprise a set of medical diagnosis labels, and the stored instructions are further executable by the at least one electronic processor 20 to perform a computer-aided diagnosis (CADx) process for a patient to be diagnosed. To do so, the stored ML decision tree classifier 12, is retrieved. The input is represented by values of the set of features for the patient to be diagnosed. A CADx diagnosis is generated by applying the retrieved ML decision tree classifier to an input represented by values of the set of features for the patient to be diagnosed. An input is received from the user via the at least one user input device 22. The generated CADx diagnosis can be displayed on the display device 24.

In another example, the decision tree classifier 12 can be used to diagnose a root cause of failure of a medical device (e.g., an imaging system). In such an example, the training data comprises medical devices represented by the set of features including medical device root cause failure features, the class labels comprise a set of root causes of the medical devices, and the stored instructions are further executable by the at least one electronic processor (20) to perform a process of determining a root cause of failure of the medical devices. To do so, the stored ML decision tree classifier 12, is retrieved. The input is represented by values of the set of features for the medical devices. For example, the features may be results of diagnostic tests, symptoms of the failure, and/or so forth. A root cause of failure of the medical device is generated by applying the retrieved ML decision tree classifier to an input represented by values of the set of features for the medical devices. An input is received from the user via the at least one user input device 22. The generated root cause of failure can be displayed on the display device 24.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a method of generating a machine-learned (ML) decision tree classifier, the method comprising:
iteratively adding child nodes to nodes of the ML decision tree classifier starting at a root node having associated training data represented by values of a set of features and labeled with class labels, wherein the addition of child nodes to a node includes:
selecting a plurality of features from the set of features;
creating one or more boundaries in a plane defined by the selected plurality of features, the one or more boundaries partitioning the plane into regions that split the training data associated to the node into at least two subsets of training data, the one or more boundaries being created based on the class labels of the training data associated to the node;
for each subset of training data, adding to the node a child node having associated training data consisting of the subset of training data; and
defining a classification rule for the node using the created one or more boundaries in the plane; and
at least one of (i) storing the ML decision tree classifier in a database and (ii) for at least one iteration of the iterative adding, displaying the plane with the created one or more boundaries on a display device.

2. The non-transitory computer readable medium of claim 1, wherein the method further includes:
generating the plane using the selected plurality of features, wherein the data in the training data associated to the node is represented in the plane as points.

3. The non-transitory computer readable medium of claim 1, wherein the plurality of features consists of two features.

4. The non-transitory computer readable medium of claim 2, wherein the creating includes:
creating the one or more boundaries as polygons in the plane.

5. The non-transitory computer readable medium of claim 4, wherein creating the one or more boundaries as polygons includes:
connecting points having different class labels in the plane with a line.

6. The non-transitory computer readable medium of claim 5, wherein the class labels include either a positive label or a negative label.

7. The non-transitory computer readable medium of claim 4, wherein creating the one or more boundaries as polygons in the plane includes:
performing a Voronoi diagram analysis on the points in the plane to generate the polygons.

8. The non-transitory computer readable medium of claim 2, wherein the creating includes:
creating the one or more boundaries as splines in the plane.

9. The non-transitory computer readable medium of claim 1, wherein the method further includes:
performing a smoothing operation on the created one or more boundaries.

10. The non-transitory computer readable medium of claim 1, wherein the selecting of the plurality of features further includes:
performing a principal component analysis on the set of training data to select the plurality of features.

11. The non-transitory computer readable medium of claim 1, wherein the method comprises (i) storing the ML decision tree classifier in the database, and the stored instructions are further executable by the at least one electronic processor to:
retrieve the stored ML decision tree classifier; and
classify an input represented by values of the set of features using the classification rules of the retrieved ML decision tree classifier.

12. The non-transitory computer readable medium of claim 1, wherein the training data comprise medical devices represented by the set of features including medical device root cause failure features, the class labels comprise a set of root causes of the medical devices, and the stored instructions are further executable by the at least one electronic processor to perform a process of determining a root cause of failure of the medical devices by:
retrieving the stored ML decision tree classifier;
generating a root cause of failure by applying the retrieved ML decision tree classifier to an input represented by values of the set of features for the medical devices; and
displaying the root cause of failure.

13. The non-transitory computer readable medium of claim 1, wherein the training data comprise patients represented by the set of features including patient features, the class labels comprise a set of medical diagnosis labels, and the stored instructions are further executable by the at least one electronic processor to perform a computer-aided diagnosis (CADx) process for a patient to be diagnosed by:
retrieving the stored ML decision tree classifier;
generating a CADx diagnosis by applying the retrieved ML decision tree classifier to an input represented by values of the set of features for the patient to be diagnosed; and
displaying the generated CADx diagnosis.

14. The non-transitory computer readable medium of claim 1, wherein the method comprises (ii) for at least one iteration of the iterative adding, displaying the plane with the created one or more boundaries on a display device, and further comprises:
providing a graphical a user interface (GUI) via which the created one or more boundaries can be adjusted to generate user-adjusted created boundaries, wherein for the at least one iteration the defining of the classification rule uses the user-adjusted created one or more boundaries.

15. An apparatus for generating a machine-learned (ML) decision classifier, the apparatus comprising at least one electronic processor programmed to:
select two features from a set of training data;
generate a plane using the selected plurality of features, wherein the data in the set of training data is represented in the plane as points;
create polygons in the plane to generate the ML decision classifier, the one or more boundaries delineating a class of the training data; and
at least one of store the ML decision classifier in a database and display the plane with the created boundaries on a display device.

16. The apparatus of claim 15, wherein the points in the plane include a label, and creating the one or more boundaries as polygons includes:
connecting points having opposing labels in the plane with a line.

17. The apparatus of claim 16, wherein the points in the plane include either a positive label or a negative label.

18. The apparatus of claim 15, wherein creating the one or more polygons in the plane includes:
performing a Voronoi diagram analysis on the points in the plane to generate the polygons.

19. The apparatus of claim 15, wherein the at least one electronic processor is further programmed to:
perform a smoothing operation on the created one or more boundaries including performing a Fourier analysis.

20. A method of generating a machine-learned (ML) decision tree classifier, the method comprising:
selecting two features from a set of training data;
generating a plane using the selected plurality of features, wherein the data in the set of training data is represented in the plane as points;
creating polygons in the plane to generate the ML decision classifier, the one or more boundaries delineating a class of the training data;
performing a smoothing operation on boundaries of the created polygons using a Fourier analysis; and
at least one of storing the ML decision classifier in a database and displaying the plane with the created boundaries on a display device.
